(19) United States
(12) Patent Application Publication
(10) Pub. No.: US 2014/0155625 A1
(43) Pub. Date: Jun. 5, 2014

(54) Modified Naturally-Derived Colorants For Phase Change Ink Applications

(71) Applicant: XEROX CORPORATION, Norwalk, CT (US)

(72) Inventors: Mihaela Maria Birau, Mississauga (CA); Salma Falah Toosi, Mississauga (CA); C. Geoffrey Allen, Waterdown (CA); Biby Esther Abraham, Mississauga (CA); Peter G. Odell, Mississauga (CA); Sandra J. Gardner, Oakville (CA)

(73) Assignee: XEROX CORPORATION, Norwalk, CT (US)

(21) Appl. No.: 13/690,857

(22) Filed: Nov. 30, 2012

(51) Int. Cl. C07D 403/04; C07C 309/44

(52) U.S. Cl. CPC C07D 403/04; C07C 309/44
USPC 548/457; 552/224

(57) ABSTRACT

A modified naturally-derived colorant comprising a naturally-derived colorant that is modified with an aliphatic quaternary ammonium salt, an aromatic quaternary ammonium salt, or a mixture or combination thereof. The modified naturally-derived colorant is compatible with phase change ink vehicles.

### RELATED APPLICATIONS

Commonly assigned U.S. patent application Ser. No. ______ (Attorney Docket number 20111073-US-NP, entitled “Phase Change Inks Containing Modified Naturally-Derived Colorants”), filed concurrently herewith, which is hereby incorporated by reference herein in its entirety, describes phase change inks including modified naturally-derived colorants.

Commonly assigned U.S. patent application Ser. No. ______ (Attorney Docket number 20111538-US-NP, entitled “Phase Change Ink Comprising Colorants Derived From Plants And Insects”), filed concurrently herewith, which is hereby incorporated by reference herein in its entirety, describes phase change inks including a modified naturally-derived colorant wherein the naturally-derived colorant is a colorant derived from a plant, a colorant derived from an insect, or a mixture or combination thereof.

### BACKGROUND

Disclosed herein is a modified naturally-derived colorant and process for preparing same, wherein the modified naturally-derived colorant comprises a naturally-derived colorant that is modified with an aliphatic quaternary ammonium salt, an aromatic quaternary ammonium salt, or a mixture or combination thereof; wherein the modified naturally-derived colorant is compatible with phase change ink vehicles.

In general, phase change inks (sometimes referred to as “hot melt inks”) are in the solid phase at ambient temperature, but exist in the liquid phase at the elevated operating temperature of an ink jet printing device. At the jet operating temperature, droplets of liquid ink are ejected from the printing device and, when the ink droplets contact the surface of the recording substrate, either directly or via an intermediate heated transfer belt or drum, they quickly solidify to form a predetermined pattern of solidified ink drops. Phase change inks have also been used in other printing technologies, such as gravure printing.

Phase change inks for color printing typically comprise a phase change ink carrier composition which is combined with a phase change ink compatible colorant. In a specific embodiment, a series of colored phase change inks can be formed by combining ink carrier compositions with compatible subtractive primary colorants. The subtractive primary colored phase change inks can comprise four component dyes, namely, cyan, magenta, yellow and black, although the inks are not limited to these four colors. These subtractive primary colored inks can be formed by using a single dye or a mixture of dyes. For example, magenta can be obtained by using a mixture of Solvent Red Dyes or a composite black can be obtained by mixing several dyes. U.S. Pat. No. 4,889,560, U.S. Pat. No. 4,889,761, and U.S. Pat. No. 5,372,852, the disclosures of each of which are totally incorporated herein by reference, teach that the subtractive primary colorants employed can comprise dyes from the classes of Color Index (C.I.) Solvent Dyes, Disperse Dyes, modified Acid and Direct Dyes, and Basic Dyes.

The colorants can also include pigments, as disclosed in, for example, U.S. Pat. No. 5,221,335, the disclosure of which is totally incorporated herein by reference.

Phase change inks have also been used for applications such as postal marking, industrial marking, and labeling.

Phase change inks are desirable for ink jet printers because they remain in a solid phase at room temperature during shipping, long term storage, and the like. In addition, the problems associated with nozzle clogging as a result of ink evaporation with liquid ink jet inks are largely eliminated, thereby improving the reliability of the ink jet printing. Further, in phase change ink jet printers wherein the ink droplets are applied directly onto the final recording substrate (for example, paper, transparency material, and the like), the droplets solidify immediately upon contact with the substrate, so that migration of ink along the printing medium is prevented and dot quality is improved.

Compositions suitable for use as phase change ink carrier compositions are known. Some representative examples of references disclosing such materials include U.S. Pat. No. 3,653,932, U.S. Pat. No. 4,390,369, U.S. Pat. No. 4,484,948, U.S. Pat. No. 4,684,956, U.S. Pat. No. 4,851,045, U.S. Pat. No. 4,889,560, U.S. Pat. No. 5,006,170, U.S. Pat. No. 5,151,120, U.S. Pat. No. 5,372,852, U.S. Pat. No. 5,496,879, European Patent Publication 0187352, European Patent Publication 0206286, German Patent Publication DE 4205636AL, German Patent Publication DE 4205713AL, and PCT Patent Application WO 94/04619, the disclosures of each of which are totally incorporated herein by reference. Suitable carrier materials can include paraffins, microcrystalline waxes, polyethylene waxes, polymethylene waxes, ester waxes, fatty acids and other waxy materials, fatty amide containing materials, sulfonamide materials, resinous materials made from different natural sources (tall oil rosins and rosin esters, for example), and many synthetic resins, oligomers, polymers, and copolymers.

Ink jetting devices are known in the art, and thus extensive description of such devices is not required herein. As described in U.S. Pat. No. 6,547,380, incorporated herein by reference, ink jet printing systems generally are of two types: continuous stream and drop-on-demand. In continuous stream ink jet systems, ink is emitted in a continuous stream under pressure through at least one orifice or nozzle. The stream is perturbed, causing it to break up into droplets at a fixed distance from the orifice. At the break-up point, the droplets are charged in accordance with digital data signals and passed through an electrostatic field that adjusts the trajectory of each droplet in order to direct it to a gutter for recirculation or a specific location on a recording medium. In drop-on-demand systems, a droplet is expelled from an orifice directly to a position on a recording medium in accordance with digital data signals. A droplet is not formed or expelled unless it is to be placed on the recording medium.

There are at least three types of drop-on-demand ink jet systems. One type of drop-on-demand system is a piezoelectric device that has as its major components an ink filled channel or passageway having a nozzle on one end and a piezoelectric transducer near the other end to produce pressure pulses. Another type of drop-on-demand system is known as acoustic ink printing. As is known, an acoustic beam exerts a radiation pressure against objects upon which it impinges. Thus, when an acoustic beam impinges on a free surface (i.e., liquid/air interface) of a pool of liquid from beneath, the radiation pressure which it exerts against the surface of the pool may reach a sufficiently high level to release individual droplets of liquid from the pool, despite the restraining force of surface tension. Focusing the beam on or near the surface of the pool intensifies the radiation pressure it exerts for a given amount of input power. Still another type of drop-on-demand system is known as thermal ink jet, or bubble jet, and produces high velocity droplets. The major components of this type of drop-on-demand system are an ink filled channel having a nozzle on one end and a heat generating resistor near the nozzle. Printing signals representing digital information originate an electric current pulse in a resistive layer within each ink passageway near the orifice or nozzle, causing the ink vehicle (usually water) in the immediate vicinity to vaporize almost instantaneously and create a bubble. The ink at the orifice is forced out as a propelled droplet as the bubble expands.

In a typical design of a piezoelectric ink jet device utilizing phase change inks printing directly on a substrate or on an intermediate transfer member, such as the one described in U.S. Pat. No. 5,372,852, incorporated herein by reference, the image is applied by jetting appropriately colored inks during four to eighteen rotations (incremental movements) of a substrate (an image receiving member or intermediate transfer member) with respect to the ink jetting head, i.e., there is a small translation of the printhead with respect to the substrate in between each rotation. This approach simplifies the printhead design, and the small movements ensure good droplet registration. At the jet operating temperature, droplets of liquid ink are ejected from the printing device and, when the ink droplets contact the surface of the recording substrate, either directly or via an intermediate heated transfer belt or drum, they quickly solidify to form a predetermined pattern of solidified ink drops.

Thermal ink jet processes are well known and are described, for example, in U.S. Pat. Nos. 4,601,777, 4,251,824, 4,410,899, 4,412,224 and 4,532,530, the disclosures of each of which are hereby incorporated herein.

Ink jet printing processes may employ inks that are solid at room temperature and liquid at elevated temperatures. Such inks may be referred to as hot melt inks or phase change inks. For example, U.S. Pat. No. 4,490,731, which is hereby incorporated by reference herein, discloses an apparatus for dispensing solid ink for printing on a substrate such as paper. In thermal ink jet printing processes employing hot melt inks, the solid ink is melted by the heater in the printing apparatus and utilized (i.e., jetted) as a liquid in a manner similar to that of conventional thermal ink jet printing. Upon contact with the printing substrate, the molten ink solidifies rapidly, enabling the colorant to substantially remain on the surface of the substrate instead of being carried into the substrate (for example, paper) by capillary action, thereby enabling higher print density than is generally obtained with liquid inks. Advantages of a phase change ink in ink jet printing are thus elimination of potential spillage of the ink during handling, a wide range of print density and quality, minimal paper cockle or distortion, and enablement of indefinite periods of nonprinting without the danger of nozzle clogging, even without capping the nozzles.

Examples of the phase change inks herein are inks that include an ink vehicle that is solid at temperatures of about 23° C. to about 27° C., for example room temperature, and specifically are solid at temperatures below about 60° C. However, the inks change phase upon heating, and are in a molten state at jetting temperatures. Thus, the inks have a viscosity of from about 1 to about 20 centipoise (cp), for example from about 5 to about 15 cp or from about 8 to about 12 cp, at an elevated temperature suitable for ink jet printing, for example temperatures of from about 60° C. to about 150° C.

In this regard, the inks herein may be either low energy inks or high energy inks. Low energy inks are solid at a temperature below about 40° C. and have a viscosity of from about 1 to about 20 centipoise such as from about 5 to about 15 centipoise, for example from about 8 to about 12 cp, at a jetting temperature of from about 60° C. to about 100° C. such as about 80° C. to about 100° C., for example from about 90° C. to about 100° C. High energy inks are solid at a temperature below 40° C. and have a viscosity of from about 5 to about 15 centipoise at a jetting temperature of from about 100° C. to about 180° C., for example from 120° C. to about 160° C. or from about 125° C. to about 150° C.

While certain colorants suitable for use in phase change inks are known, an increase in the range of colorants suitable for use in phase change inks is desirable.

U.S. patent application Ser. No. 13/008,783, filed Jan. 18, 2011, of Maria Birau, et al., entitled “Phase Change Ink Compositions And Colorants For Use In The Same,” which is hereby incorporated by reference herein in its entirety, describes in the Abstract thereof phase change ink compositions comprising a novel colorant wax to prevent and/or reduce print head and nozzle contamination in ink jet printers caused by drooling and faceplate staining. In particular, there is provided novel colorants containing acid groups for use in phase change ink compositions and which are compatible with phase change ink components.

Natural colorants, for example, Alizarin and Indigo, are desirable as bio-friendly or “green” colorants, but are not suitable for use in phase change ink vehicles due to the difficulty of dispersing such colorants owing to their large particle size and lack of functionality to enable dispersant attachment to the pigment particle. Sulfonated analogs of naturally-derived colorants contain functionality but are typically difficult to disperse in low polarity solid ink vehicles. Further, residual salts from these compounds can precipitate out from the ink in the print head and form undesirable salt rings around the ink jet orifices thus hindering reliable jetting performance.

While known compositions and processes are suitable for their intended purposes, a need remains for improved colorants, and more specifically, for improved colorants suitable for use in phase change inks. Additionally, a need remains for improved colorants suitable for use in phase change inks that are natural or derived from natural sources and thus environmentally friendly.

The appropriate components and process aspects of the each of the foregoing U.S. Patents and Patent Publications may be selected for the present disclosure in embodiments thereof. Further, throughout this application, various publications, patents, and published patent applications are referred to by an identifying citation. The disclosures of the publications, patents, and published patent applications referenced in this application are hereby incorporated by reference into the present disclosure to more fully describe the state of the art to which this invention pertains.

### SUMMARY

Described is a modified naturally-derived colorant comprising a naturally-derived colorant that is modified with an aliphatic quaternary ammonium salt, an aromatic quaternary ammonium salt, or a mixture or combination thereof. In embodiments, the modified naturally-derived colorant is compatible with phase change ink vehicles.

Also described is a process for preparing a modified naturally-derived colorant comprising providing a naturally-derived colorant; contacting the naturally-derived colorant with a modifying component selected from the group consisting of an aliphatic quaternary ammonium salt, an aromatic quaternary ammonium salt, or a mixture or combination thereof; to provide a modified naturally-derived colorant that is compatible with phase change ink vehicles.

### BRIEF DESCRIPTION OF THE DRAWINGS

FIG. 1 is a transmission electron micrograph image showing non-modified indigo carmine.

FIG. 2 is a transmission electron micrograph image showing modified indigo carmine in accordance with an embodiment of the present disclosure.

### DETAILED DESCRIPTION

A modified naturally-derived colorant comprising a naturally-derived colorant that is modified with an aliphatic quaternary ammonium salt, an aromatic quaternary ammonium salt, or a mixture or combination thereof; wherein the modified naturally-derived colorant is compatible with phase change ink vehicles is described. In embodiments, the naturally-derived colorant is a pigment, a dye, or a mixture or combination thereof. In certain embodiments, the naturally-derived colorant is a pigment. In other embodiments, the naturally-derived colorant is a dye.

Natural, naturally-derived or “natural like” colorant as used herein can be described as colorants that exist in nature but having a structure which has been functionalized to add functional groups that the original structure does not have or a synthetically prepared compound that has the structure of a naturally occurring colorant. Some natural or naturally-derived colorants including pigments and dyes are known for their thermal stability and good light fastness. It is desirable to be able to use natural or naturally-derived colorants in solid ink.

Natural colorants (consisting of dyes and pigments) can be derived from natural sources all over the world including from plants, animals and microorganisms such as bacteria that extract the colorant of various materials. Most natural colorants fall into 6 general classes which include tetrapyrroles, tetra-terpenoids, quinines, O-heterocyclic compounds, N-heterocyclic compounds and metallo-proteins. Examples of tetrapyrroles include porphyrins and porphyrin derivatives and more specifically, chlorophylls, heme pigments and bilins. Examples of tetra-terpenoids or carotenoids include carotenes and xanthophylls. Examples of quinines include benzoquinones, anthraquinones and naphthiquinones. Examples of O-heterocyclic compounds such as flavonoids include anthocyanins and flavonols. Examples of N-heterocyclic compounds include indigoids and indole derivatives, such as betalaines and eumelanins, and substituted pyrimidines such as pterins and purines. Examples of metallo-proteins, such as oligomeric proteins, include iron-based proteins such as haemerythrin and myohemerythrin which exhibit color in an oxygenated state. Other examples of natural colorants include lipofuscins and fungal pigments. In embodiments herein, the naturally-derived colorant is derived from a member of the group consisting of tetrapyrroles, tetra-terpenoids, quinines, O-heterocyclic compounds, N-heterocyclic compounds, metallo-proteins, lipofuscins, and fungal pigments.

Natural colorants have or can be made to have a variety of colors with or without metal complexing agents such as magnesium and iron and/or with or without the use of a mordant such as alum, tin, stannous chloride, copper sulfate and the like. Some natural colorants are prone to oxidation which can result in the natural colorant having a different color before it was oxidized. Described herein are examples of mixtures of modified naturally derived pigments and dispersants that when incorporated into phase-change inks impart a grayish or metal-like appearance on a print such as a print on white paper.

Examples of red dyes from natural sources include those from Safflower (Carthamus tinctorius), Caesalpina (Caesalpina sappan), Maddar (Rubia tinctorum), Kermes (Kermes vermilio), Drago tree (Dracaena draco), Daemonorops (Daemonorops draco), Cochineal (Dactylopiuscoccus coccus) and Lac (Coccus lacca). Examples of yellow dyes from natural sources include those from Bougainvillea (Bougainvillea glabra), Golden rod (Solidago grandis), Teak (Tectona grandis), Marigold (Tagetes species), Weld (Reseda luteola), Saffron (Crocus sativus) and Parijata (Nyetanthasar bortristis). Examples of blue or blue-like (such as purple) dyes from natural sources include those from Indigo (Indigofera tinctoria), Woad (Isatis tinctoria), Suntberry (Acacia nilotica), Pivet (Ligustrum vulgare), molluscs such as Bolinus brandaris, Hexaplex trunculus and Stramonita haemastoma, Murasaki (Lithospermum erythrorhizon) and Water lily (Nymphaea alba). Examples of green dyes from natural sources include those from Tulsi (Ocimum sanctum), Bougainvillea (Bougainvillea glabra), Canna, Lily (Convallaria majalis) and Nettles (Urtica diocia). Examples of orange or orange-like dyes from natural sources include those from Bougainvillea (Bougainvillea glabra), Balsam (Impatiens balsamina), Dahlia (Dahlia species) and Annatto (Bixa orellana). Examples of brown dyes from natural sources include those from Caesalpina (Caesalpina sappan), Bougainvillea (Bougainvillea glabra), Balsam (Impatiens balsamina), Marigold (Tagetes species), Balsam (Impatiens balsamina) and Blackberries (Rubus fructicosus). Examples of black dyes from natural sources include those from Lac (Coccus lacca), Alder (Almus glutinosa), Rofblamala (Loranthus pentapetalus), Custard apple (Anona reticulata) and Harda (Terminalia chebula). In embodiments, the naturally-derived colorant is derived from a member of the group consisting of Safflower, Caesalpina, Maddar, Kermes, Drago tree, Daemonorops, Cochineal, Lac, Bougainvillea, Golden rod, Teak, Marigold, Weld, Saffron, Parijata, Indigo, Woad, Suntberry, Pivet, molluscs, Murasaki, Water lily, Tulsi, Canna, Lily, Nettles, Balsam, Dahlia, Annatto, Balsam, Blackberries, Lac, Alder, Rofblamala, Custard apple, Harda, and mixtures and combinations thereof.

To achieve a desired color or effect, two or more natural colorants may be mixed together in any proportion to achieve that desired color or effect. It is also known that two or more natural colorants may be present in a given natural colorant compound.

Natural colorants such as indigo, referenced as C.I. Pigment Blue 66, C.I. Vat Blue 1 and C.I. Reduced Vat Blue 1 (all listed as C.I. 73000) and its variants thereof, and alizarin, referenced as C.I. Mordant Red 11 (listed as C.I. 58000) and their variants thereof are known as “ancient blues” and “ancient reds”, respectively, and played important roles as colorants for several ancient civilizations. In embodiments herein, various naturally-derived pigments, such as indigo and alizarin, were derivatized such that they became compatible and dispersible within phase change ink vehicles.

The natural or naturally-derived colorants can be obtained from natural sources or obtained commercially. Naturally-derived colorants herein can include colorants that were originally natural or bio-based colorants that were modified from their natural state but not suitable as modified for use in phase change inks. These naturally-derived colorants can be modified as described herein to provide modified naturally-derived colorants suitable for use in phase change inks.

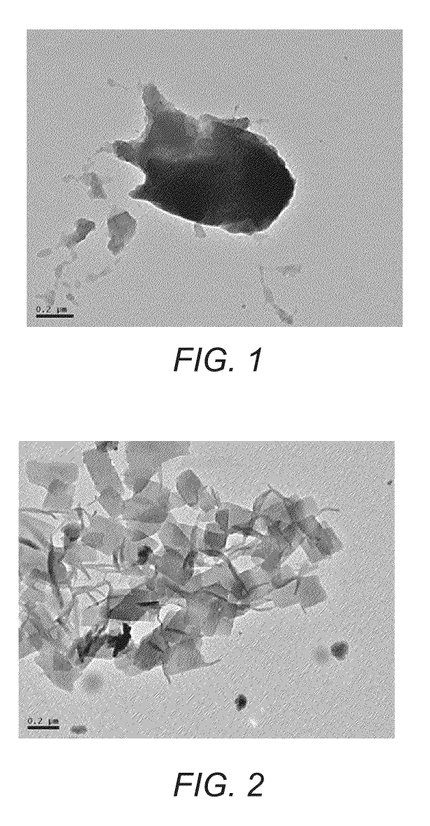

In embodiments, a naturally-derived colorant, in embodiments, a pigment, that had been modified to contain a sodium atom (or any other Group I alkali metal, especially potassium) was modified in accordance with the present disclosure to replace the sodium atom with a long chain alkyl quaternary ammonium salt. In embodiments, this modification provided a reduction in the particle size of the pigment. In embodiments, modifying Indigo Carmine with N,N-dimethyldioctadecylammonium bromide provided a reduction in the pigment particle size from an original particle size of more than about 2 micrometers to an after-modification particle size of less than about 300 nanometers in length (or long axis or longest dimension of particle) as determined by Transmission Electron Microscopy (TEM). FIG. 1 shows a TEM picture of non-modified indigo carmine. FIG. 2 shows a TEM picture of indigo carmine modified with N,N-dimethyldioctadecylammonium bromide. Non-modified indigo pigments are characterized by irregular shaped agglomerates of poorly defined primary particles. These agglomerates are typically in excess of about 2 micrometers in diameter. The modified indigo pigments of the present disclosure are characterized by well-defined rectangular-shaped primary particles ranging from about 150 nanometers to about 200 nanometers along the long axis of the particle.

The inventors believe that the present modified naturally-derived pigments are novel compounds. The modified naturally-derived colorants, in embodiments, modified naturally-derived pigments, are suitable for use in phase change ink vehicles and can be used to develop spot and secondary colors, such as orange and violet, although not limited to these colors.

In embodiments, the modified naturally-derived colorant herein is derived from indigo of the formula

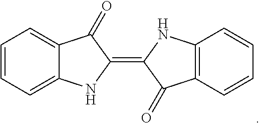 [SMILES: O=C1/C(=C2\Nc3ccccc3C2=O)Nc2ccccc21]

In other embodiments, the modified naturally-derived colorant can also be derived from indirubin (also known as Indigo Red (CAS No. 75790)) of the formula

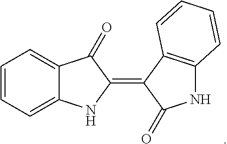 [SMILES: O=C1Nc2ccccc2/C1=C1/Nc2ccccc2C1=O]

In still other embodiments, the modified naturally-derived colorants can also be derived from indigoids and indirubinoids including those of the formulae

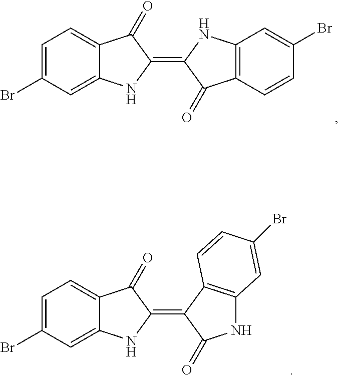 [SMILES: O=C1/C(=C2\Nc3cc(Br)ccc3C2=O)Nc2cc(Br)ccc21 ; O=C1Nc2cc(Br)ccc2/C1=C1/Nc2cc(Br)ccc2C1=O]

In other embodiments, the modified naturally-derived colorant herein is derived from alizarin or anthraquinone red of the formula

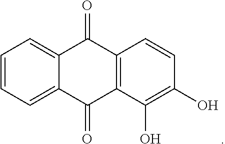 [SMILES: O=C1c2ccccc2C(=O)c2c1ccc(O)c2O]

In other embodiments, the modified naturally-derived colorant can also be derived from Alizarin purpurin (referenced as C.I. Natural Red 8 and C.I. Natural Red 16 and listed as C.I. 75410 and also known as Indigo Red) of the formula

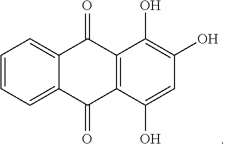 [SMILES: O=C1c2ccccc2C(=O)c2c(O)c(O)cc(O)c21]

Included in further embodiments, the modified naturally-derived colorants can also be derived from naturally occurring anthraquinoids such as in Table 1 as related to the formula

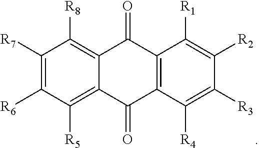

TABLE 1
Chemical Structures of Naturally Occurring Anthraquinoids

| Dye | Substituents | | | | | | | |
|---|---|---|---|---|---|---|---|---|
| Component | R₁ | R₂ | R₃ | R₄ | R₅ | R₆ | R₇ | R₈ |

Madder Plant

| | | | |
|---|---|---|---|
| Alizarin | OH | OH | |
| Purpurin | OH | OH | OH |

Insects

| | | | | | | | |
|---|---|---|---|---|---|---|---|
| Carminic | CH₃ | COOH | OH | OH | OH | (c) | OH |
| acid | | | | | | | |
| Flavo- | CH₃ | COOH | OH | | OH | | OH |
| kermesic | | | | | | | |
| acid | | | | | | | |
| Kermesic | CH₃ | COOH | OH | OH | OH | | OH |
| acid | | | | | | | |
| Laccaic | COOH | COOH | OH | OH | OH | (a) | OH |
| acid A | | | | | | | |
| Laccaic | COOH | COOH | OH | OH | OH | (b) | OH |
| acid B | | | | | | | |

The (a), (b) and (c) designations in Table 1 refer to the following formulae

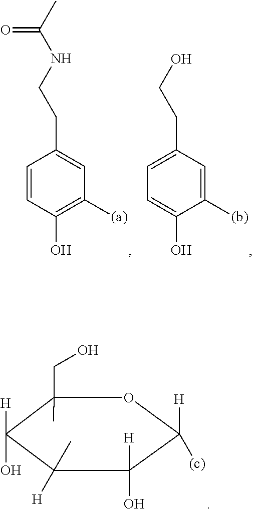

The modified naturally-derived colorants herein can be derived from sulfonated compounds. In embodiments, the present modified naturally-derived colorants are derived from sulfonated indigo carmine having sodium counter ions (also known as Indigotine) of the formula

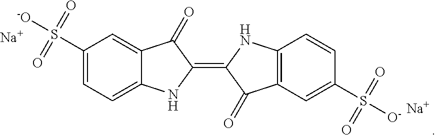

Other embodiments include the modified naturally-derived colorants derived from a sulfonated indigo known as disodium 2-(1,3-dihydro-3-oxo-7-sulphonato-2H-indol-2-ylidene)-3-oxoindoline-5-sulphonate (CAS No. 27414-68-2) of the formula

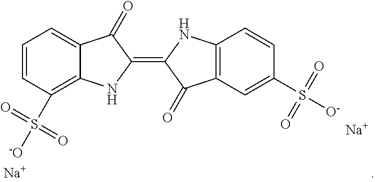

In other embodiments, the modified naturally-derived colorants can also include indigo pigments having degrees of sulfonation of 3 or 4 and having potassium counterions of the formulae

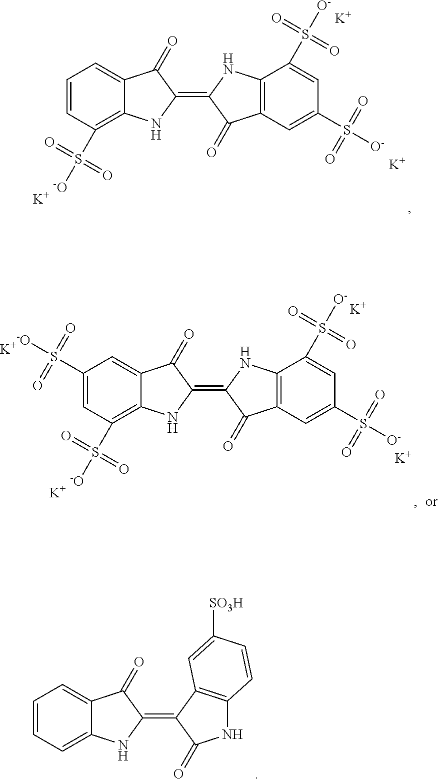

In yet other embodiments, the modified naturally-derived colorants can be derived from sulfonated indirubin including Indirubin-5-sulfonic acid (CAS No. 864131-82-8) and salts of same where the free acid has the formula

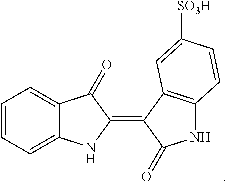 [SMILES: O=C1Nc2ccc(S(=O)(=O)O)cc2/C1=C1/Nc2ccccc2C1=O]

In other embodiments, the present modified naturally-derived colorants are derived from sulfonated Alizarin Red S having a sodium counter ion of the formula

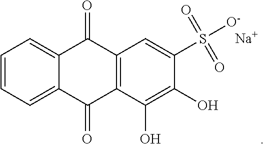 [SMILES: O=C1c2ccccc2C(=O)c2c1cc(S(=O)(=O)[O-])c(O)c2O ; [Na+]]

In other embodiments, the modified naturally-derived colorants can be derived from Purpurin sulfonate (CAS No. 6486-90-4) having a sodium counter ion of the formula

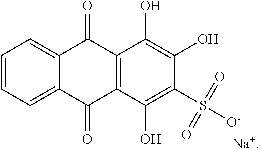

Sulfonated compounds are commercially available, for example, under the name of “Indigo Carmine” and “Alizarin Red S” from Sigma-Aldrich®.

In other embodiments, the sulfonates' counterions of the naturally-derived colorants can be sodium and/or potassium.

The modified naturally-derived colorants herein include a modifying component that renders the natural or naturally-derived colorants suitable for use in solid ink. In embodiments, certain naturally-derived colorants are modified by replacing the sodium therein with a quaternary ammonium alkylated or olephenic group which renders the modified naturally-derived colorant dispersible in in solid ink vehicles and further eliminates the risk of salt precipitation inside the print head.

Any suitable or desired aliphatic, olephenic, or aromatic quaternary salt, or mixture or combination thereof can be selected in embodiments herein.

In embodiments, the naturally-derived colorant is modified with a compound of the formula

R—N⁺(CH₃)₃X⁻

wherein R is a long chain alkyl group having at least 8 carbon atoms and X is a halogen.

In embodiments, the modified naturally-derived colorants herein include a modifying component that is an aliphatic quaternary ammonium salt or aromatic quaternary ammonium salt and mixtures thereof and any suitable halide such as chlorine, bromine or iodine. Suitable N-alkyl/aryl counterions to be used in the modification may be selected from the group consisting of quaternary ammonium NH₄, or any alkyl or aryl quaternary ammonium, such as tetrabutylammonium, tetraoctylammonium, tetradodecylammonium, tetraoctadecylammonium, N,N-dimethyl dioctadecylammonium, N,N-dimethyl dioctyl ammonium, N,N-dimethyl dodecyl ammonium, N,N,N-trimethyl-1-docosanaminium, behenyl trimethylammonium, N-octadecyltrimethylammonium, and other quaternary ammonium compounds such as the quaternary ammonium compounds known as ARQUAD®s available from Akzo Nobel N.V., and mixtures thereof.

The quaternary ammonium compounds known as the ARQUAD®s are primarily alkyltrimethylammonium chlorides and may be represented by the formula R—N(CH₃)₃Cl wherein R is a long chain alkyl group having at least 8 carbon atoms. These particular quaternary ammonium compounds are marketed by Akzo Nobel N.V. under the trade-name ARQUAD®. Examples of suitable ARQUAD® materials are: Arquad® 316, cocoalkyltrimethylammonium from ARQUAD® C-35, didecydimethylammonium from ARQUAD® 2.10-50, ARQUAD® 2.10-70 HFP, 2.10-80, coco(fractionated) dimethylbenzylammonium from ARQUAD® MCB 33, ARQUAD® MCB 50, ARQUAD® MCB 80, hexadecyltrimethylammonium from ARQUAD® 16-29, stearyltrimethylammonium from ARQUAD® 18-50, behenyltrimethylammonium from ARQUAD® 20-80, or salts thereof. A variety of compounds of this class are available varying as to the length and number of long chain alkyl groups attached to the nitrogen atom. Compounds are understood to have appropriate charge on counter ion whether or not charge on counter ion is shown. In other embodiments, the N-alkyl or N-aryl counterion is selected from one of the following:

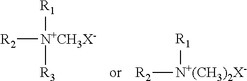

wherein R₁, R₂ and R₃ can be identical or different from one another and wherein each of R₁, R₂ and R₃ is independently selected from the group consisting of alkyl, alkoxy, aryl, and alkylaryl and wherein X is any halogen atom. In embodiments, the alkyl, alkoxy, aryl, and alkylaryl groups have equal to or greater than 4 carbon atoms. The quaternary ammonium counter ion can also include alkoxylates such as the following:

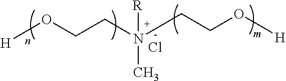

wherein R is H, CH₃, any alkyl linear or branched, or alkoxyl; and wherein m is an integer from 1 to 25 and wherein n is an integer from 1 to 25 and wherein, in embodiments m+n is from about 2 to about 25.

Examples include Ethoquad® C/12 wherein R is coco (a complex mixture of unsaturated and saturated C6 to C18 acids from coconut oil) and wherein m+n=2, Ethoquad® C/25 wherein R is coco and wherein m+n=15, Ethoquad® O/12 wherein R is oleyl and m+n=2, all available from Lion Akzo Corporation.

In embodiments, the quaternary ammonium counter ion herein can be an oligomer of the general formula

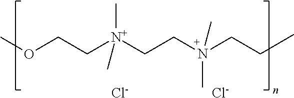

wherein n is an integer, in embodiments, wherein n is at least 1, in embodiments, wherein n is from about 1 to about 30. In a specific embodiment, the quaternary ammonium counter ion is poly[oxy-1,2-ethanediyl(dimethyliminio)-1,2-ethanediyl(dimethyliminio)-1,2-ethanediylchloride (1:2)]. A polixetonium chloride is commercially available from Advantis Technologies, Inc.

Examples of counter ions containing aryl groups include, but are not limited to, benzyltributylammonium bromide, benzyltributylammonium chloride, benzyltriethylammonium chloride, benzyltriethylammonium bromide, benzyltriethylammonium iodide, benzyltrimethylammonium iodide, benzyltrimethylammonium bromide, benzyltrimethylammonium chloride (neat or in solution)].

In certain embodiments, the naturally-derived colorant is modified with an aliphatic quaternary ammonium salt comprising an alkyl chain having at least eight carbon atoms, and, in embodiments, having more than 8 carbon atoms.

In other embodiments, the aliphatic quaternary ammonium salt, aromatic quaternary ammonium salt, or a mixture or combination thereof, is bio-based. Bio-based as used herein means that the aliphatic quaternary ammonium salt, an aromatic quaternary ammonium salt, or a mixture or combination thereof, is derived from natural sources. Bio-based materials are materials that are derived in whole or part from renewable biomass resources. Biomass resources are organic materials that are available on a renewable or recurring basis such as from crop residues, wood residues, grasses, and aquatic plants and as derived from bacteria and other microorganisms. Corn ethanol is a well-known example of a bio-based material derived from biomass resources. A bioproduct is a product that contains some amount of biobased material within it.

In other embodiments, the modified naturally-derived colorant is modified with an aliphatic quaternary ammonium salt, aromatic quaternary ammonium salt, or mixture or combination thereof, that contains an alkyl chain having at least 8 carbon atoms, and in embodiments having more than 8 carbon atoms, wherein the alkyl chain is biobased. Bio-based as used here means that the alkyl chain portion of the aliphatic quaternary ammonium salt, aromatic quaternary ammonium salt, or mixture or combination thereof is derived from natural sources.

Examples of bio-based quaternary ammonium salts include, but are not limited to, polyquaternium-4 (hydroxyethyl cellulose dimethyl diallylammonium chloride copolymer), polyquatemium-10 (quaternized hydroxyethyl cellulose), ARQUAD® PC 268-75 PG (ceteardimonium chloride and propylene Glycol), ARQUAD® PC C-33W (cocotrimonium chloride), ARQUAD® PC C-35 (cocotrimonium chloride), ARQUAD® PC 2C-75 (dicocodimonium chloride (and) isopropyl alcohol), ARQUAD® PC 16-29 (cetrimonium chloride), ARQUAD® PC 16-50 (cetrimonium chloride and isopropyl alcohol), ARQUAD® C-33W (cocoalkyl ammonium chloride), ARQUAD® C-50 (cocoalkyl ammonium chloride), ARQUAD® S-50 (soyaalkylammonium chloride), ARQUAD® T-27W (tallowalkylammonium chloride), ARQUAD® T-50 (tallowalkylammonium chloride), ARQUAD® 2C-70 nitrite dicocoalkyl-(b)-ammonium chloride), ARQUAD® 2C-70 PG (dicocoalkylammonium chloride), ARQUAD® 2C-75 (dicocoalkylammonium chloride), ARQUAD® 2HT-75 di(hydrogenated tallowalkyl)ammonium chloride), ARQUAD® 2HT-75 PG di(hydrogenated tallowalkyl)-(a)-ammonium chloride), ARQUAD® HTL8 MS 2-ethylhexyl hydrogenated tallowalkyl-(c)-ammonium chloride), ARQUAD® DMCB-80 (benzyldimethyl-cocoalkylammonium chloride), ARQUAD® DMHTB-75 (benzyldimethyl-(hydrogenated tallowalkyl)ammonium chloride), ARQUAD® M2HTB (benzylmethyl-di(hydrogenated tallowalkyl)ammonium chloride), ETHOQUAD® C/12B (benzylcocoalkyl[ethoxylated (2)]-ammonium chloride), ETHOQUAD® C/12-75 (cocoalkylmethyl[ethoxylated (2)]-ammonium chloride), ETHOQUAD® C/12 Nitrate (cocoalkylmethyl[ethoxylated (2)]-ammonium nitrate, ETHOQUAD® C/25 (cocoalkylmethyl[ethoxylated (15)]-ammonium chloride, ETHOQUAD® O/12 PG (oleylmethyl[ethoxylated (2)]-ammonium chloride, ETHOQUAD® T/13-27W (tris(2-hydroxyethyl)tallowalkylammonium acetates, ETHOQUAD® T/25 (tallowalkylmethyl)ethoxylated (15)1-ammonium chloride, DUOQUAD® T-50 (N,N,N′,N′,N′-pentamethyl-N-tallow-1,3-propanediammonium dichloride and the like and mixtures thereof.

In specific embodiments, the modifying component is a quaternary ammonium salt such as N,N-dimethyldioctadecyl ammoniumbromide.

In specific embodiments, the quaternary ammonium counter ion herein can be esterquats of the formulae

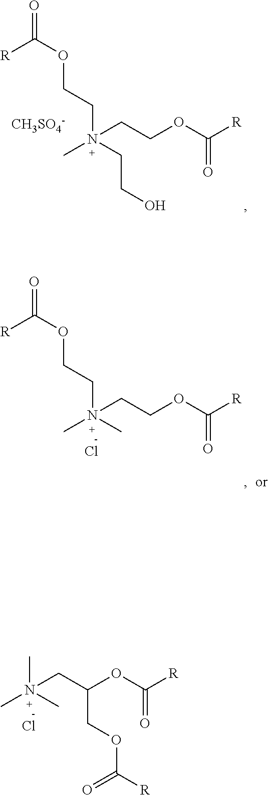

wherein R is n-heptyl, n-nonyl, n-undecyl, n-tridecyl, n-pentadecyl, n-heptadecyl, n-nonadecyl, n-heneicosyl and mixtures thereof.

Examples of esterquats include, but are not limited to, esterquats such as those available from Kao Chemicals Inc., quaternary ammonium salts of: reacted fatty acids, C10-C20 and unsaturated C16-C18, with triethanolamine (CAS No. 91995-81-2), reacted tallow fatty acids with triethanolamine (CAS No. 93334-15-7), reacted fatty acids, C12-C20 with triethanolamine (CAS No. 91032-11-0), reacted 9-octadecenoic acid (Z) with triethanolamine (CAS No. 94095-35-9), reacted octadecenoic acid with triethanolamine (CAS No. 85408-12-4). Other examples of esterquats include dimethylbis [2-[(1-oxooctadecyl)oxy]ethyl]ammonium chloride (CAS No. 67846-68-8), Dimethylbis [2-[(1-oxohexadecyl)oxy]ethyl]ammonium chloride (97158-31-1) and (Z)-2-hydroxy-3-[(1-oxo-9-octadecenyl)oxy]propyltrimethylammonium chloride (CAS No. 19467-38-0).

In other embodiments, quaternary ammonium compounds comprising alkyltrimethylammonium chlorides represented by the formula R—N(CH₃)₃C1 wherein R is a long chain alkyl group having at least 8 carbon atoms can be selected as the modifying component. A variety of compounds of this class are available varying as to the length and number of long chain alkyl groups attached to the nitrogen atom. Certain quaternary ammonium compounds are marketed by Akzo Nobel N.V. under the trade-name ARQUAD® such as Arquad® 316.

In specific embodiments, the modified naturally-derived colorant is of the formula

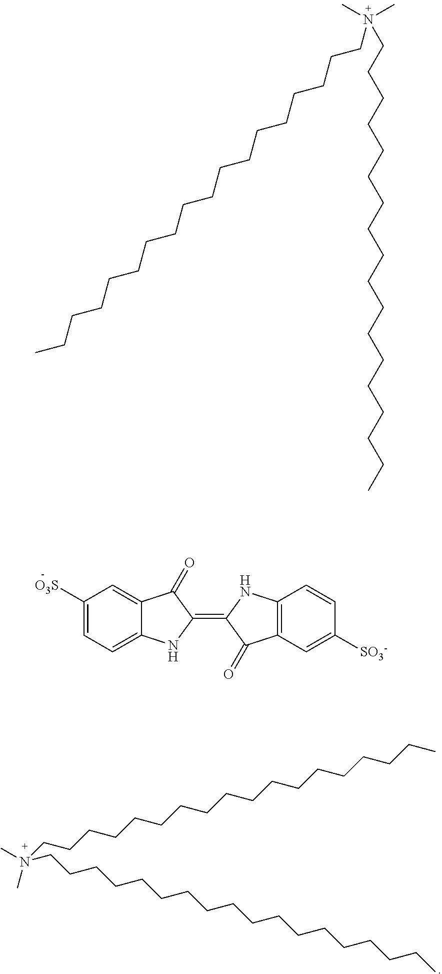

In further specific embodiments, the modified naturally-derived colorant is of the formula

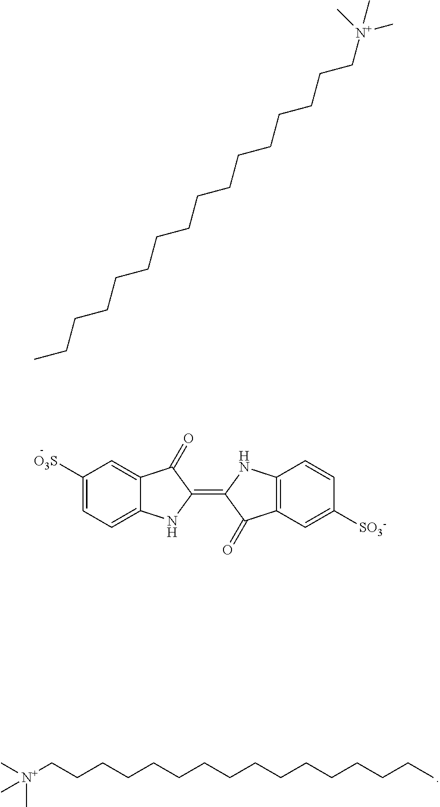

In further specific embodiments, the modified naturally-derived colorant is of the formula

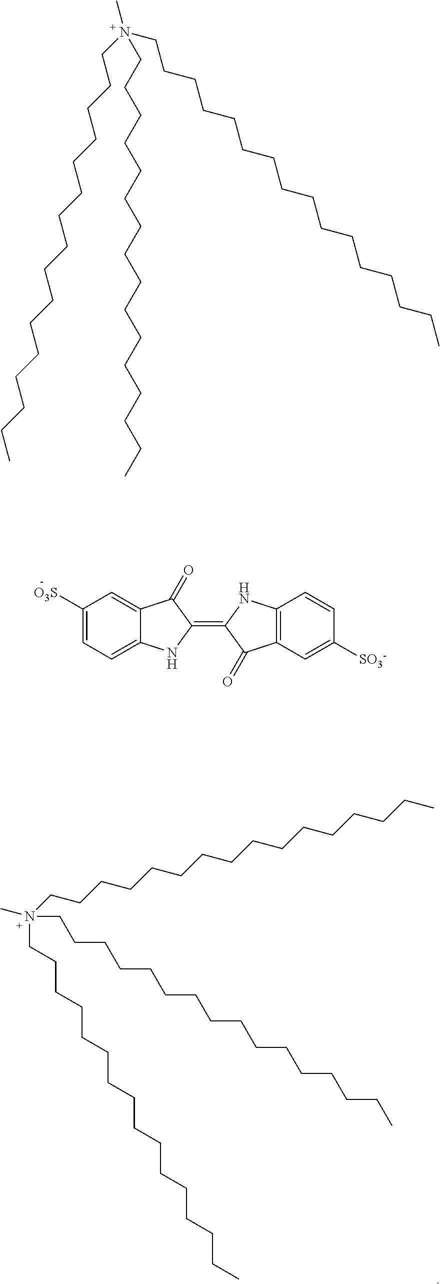

In other specific embodiments, the modified naturally-derived colorant is of the formula

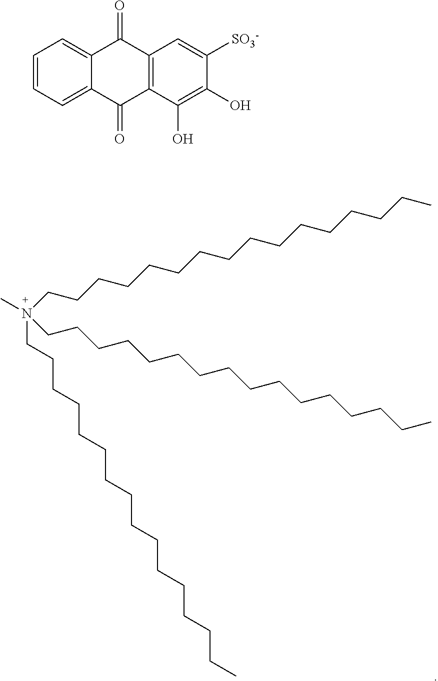

The modified naturally-derived colorants herein can be prepared by any suitable or desired process. In embodiments, a process for preparing a modified naturally-derived colorant in accordance with the present disclosure comprises providing a natural or naturally-derived colorant; contacting the naturally-derived colorant with a modifying component selected from the group consisting of an aliphatic quaternary ammonium salt, an aromatic quaternary ammonium salt, or a mixture or combination thereof; to provide a modified naturally-derived colorant that is compatible with phase change ink vehicles. In embodiments, the naturally-derived colorant contains a sodium counter ion and the modifying component displaces the sodium counter ion.

Contacting the naturally-derived colorant with a modifying component can comprise contacting in water with heating, and, optionally, isolating the modified naturally-derived colorant.

Any suitable or desired amount of naturally-derived colorant and modifying component can be used. In embodiments, the naturally-derived colorant and modifying component are provided in a 1:1 ratio wherein the naturally derived colorant contains one sulfonate group such as a modified alizarin pigment including Alizarin Red S.

In other embodiments, the naturally-derived colorant and modifying component are provided in a 1:2 ratio wherein the naturally derived colorant is a modified indigo pigment containing two sulfonate groups such as a modified indigo pigment including C.I. Acid Blue 74.

In still other embodiments, the naturally-derived colorant and modifying component are provided in a 1:3 ratio wherein the naturally derived colorant is a modified indigo pigment containing three sulfonate groups such as 2-(1,3-dihydro-3-oxo-5-sulfo-2H-indol-2-ylidene)-2,3-dihydro-3-oxo-, tripotassium salt (CAS No. 67627-18-3) represented by the structure

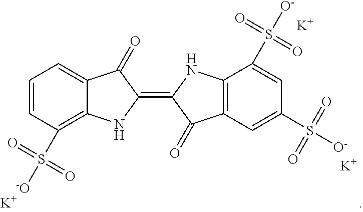

In yet other embodiments, the naturally-derived colorant and modifying component are provided in a 1:4 ratio wherein the naturally derived colorant is a modified indigo pigment containing four sulfonate groups such as a modified indigo pigment including the structure

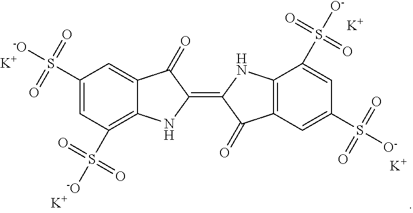

Any suitable or desired amount of water can be used. In embodiments, the naturally-derived colorant and a modifying component are contacted in about 100 to about 1,000 milliliters of water. The amount of water can be selected by one of skill in the art determined by the reaction scale. For larger scales, more water is necessary. Typically, 1 gram of colorant to approximately 34 grams of water is selected. Before modification, the colorant is soluble in water; once the modification is completed, the compound becomes insoluble in water and can be easily isolated.

The naturally-derived colorant with a modifying component can be heated to any suitable or desired temperature. In embodiments, heating can be to from about 40° C. to about 100° C., or from about 50° C. to about 90° C., or from about 60° C. to about 80° C., for a period of from about 1 to about 6 hours, or from about 2 to about 5 hours, or from about 3 to about 4 hours. Any removal of water from the original amount used, such as by evaporation due to heating, can be reinstated with a suitable addition of more water.

The final modified naturally-derived colorant product can be isolated, such as by filtration, and optionally washed, such as with water, to remove inorganic salts.

In some embodiments, such as Indigo Carmine, the pigment character has been preserved in the modified compound such that the modified compound is insoluble in solvents and water. In such embodiments, a dispersant can be employed for solid ink stabilization.

The present modification has been very beneficial for particle size reduction. In embodiments, the particle size of the final product has been reduced from an original particle size of typically about 1 micrometer to about 200 nanometers volume average particle diameter as determined by Transmission Electron Microscopy. FIG. 1 shows a micrograph of the non-modified Indigo Carmine FIG. 2 shows a micrograph of an Indigo Carmine of the present disclosure as modified with N,N-dimethyldioctadecylammonium bromide.

In embodiments, the modified-naturally-derived colorants herein are self-dispersible in phase change ink vehicles. In specific embodiments, modified Alizarin Red S gained a “dye-like” character including the ability to self-disperse in solid ink.

### EXAMPLES

The following Examples are being submitted to further define various species of the present disclosure. These Examples are intended to be illustrative only and are not intended to limit the scope of the present disclosure. Also, parts and percentages are by weight unless otherwise indicated.

Commercially available colorants (Alizarin Red S, Indigo), N,N-dimethyl dioctadecyl bromide were purchased from Sigma-Aldrich®. Arquad® 316 was obtained from Akzo Nobel. Alizarin Red S was modified using Arquad® 316, and the yield for the modified compound (COMPOUND 4) varies between 49% and 89% by weight. Indigo Carmine was modified using N,N-dimethyldioctadecyl ammoniumbromide and the yield for the modified compound (COMPOUND 1) was about 90% by weight. Indigo Carmine was also modified using cetyltrimethylammonium bromide and the yield for the modified compound (COMPOUND 2) was about 92%. Indigo Carmine was modified with Arquad® 316 and the yield for the modified compound (COMPOUND 3) was about 90%. The structure of the modified colorants is presented in Table 2 below:

TABLE 2

| Ex- | | | Counter | |
|---|---|---|---|---|
| ample | | | ion | Yield |
| # | Name | Structure | used | (%) |
| 1 | COM- POUND 1 | 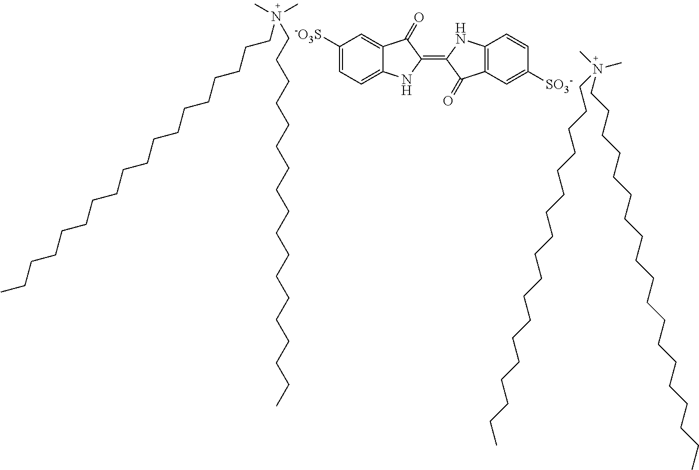 | N,N- dimethyl- diocta- decyl ammo- nium | 90 |
| 2 | COM- POUND 2 | 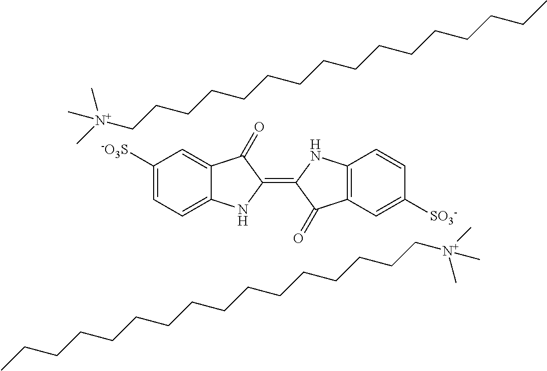 | Cetyltri- methyl- ammo- nium | 92 |
| 3 | COM- POUND 3 | 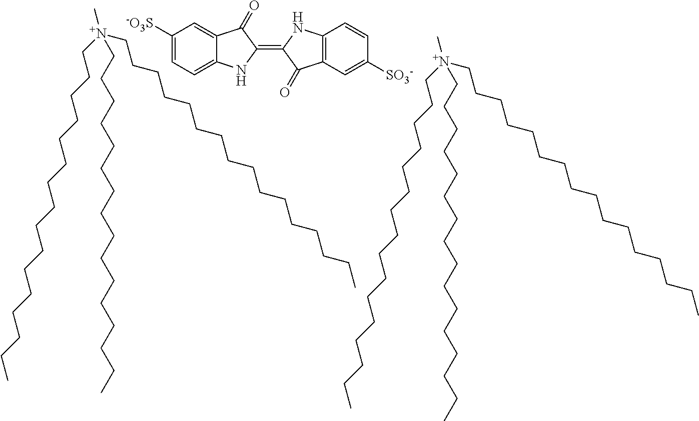 | Ar- quad ® 316 | 75 |
| 4 | COM- POUND 4 | 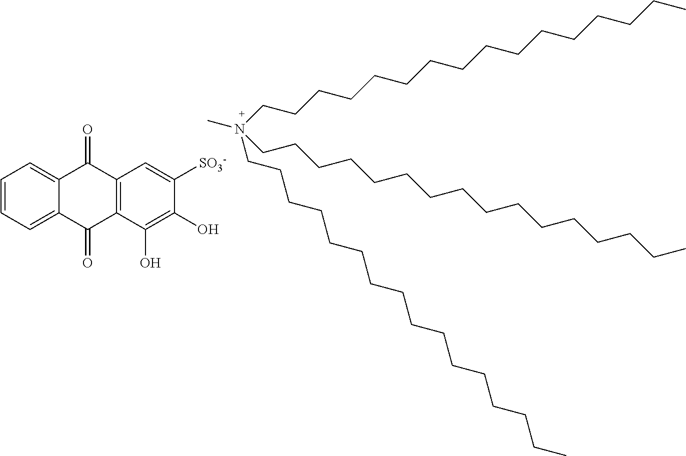 | Ar- quad ® 316 | 49 |

The ink components are presented in Table 3. The percentages in the ink formulation are based on weight.

TABLE 3

| Ink Base Component | Details |
|---|---|
| Polymethylene wax | A fractionated polymethylene wax |
| | available from IGI Inc. |
| Triamide wax | As prepared in Example 1 of U.S. |
| | Pat. No. 6,860,930 |
| Kemamide ® S-180 | Stearyl stearamide available from |
| | Witco Chemical Corporation |
| KE-100 | an ester of tetrahydroabietic acid |
| | and glycerol available from |
| | Arakawa Industries |
| Urethane resin | As prepared in example 4 of U.S. |
| | Pat. No. 6,309,453 |
| Naugard ® 445 | Antioxidant available from Uniroyal |
| | Chemical Company |

Also used in the examples is a dispersant prepared as described in Example 1 of U.S. Pat. No. 7,973,186, which is hereby incorporated by reference herein in its entirety, a dispersant prepared as described in Example 4 of U.S. Pat. No. 6,309,453, which is hereby incorporated by reference herein in its entirety, a triamide wax as described in Example II of U.S. Pat. No. 6,860,930, which is hereby incorporated by reference herein in its entirety, and Solsperse® 17000, available from The Lubrizol Corporation.

A dispersant prepared as described in Example 1 of U.S. Pat. No. 7,973,186, is prepared as follows. Into a 1 liter resin kettle fitted with heating mantle, mechanical stirring, Dean-Stark trap, reflux condenser and temperature sensor were introduced 192.78 grams (g) of Unicid® 700 (a long chain, linear carboxylic acid having an average carbon chain length of 48, available from Baker Petrolite) and 60.3 g of E-100® (a mixture of tetraethylenepentamine, (TEPA), pentaethylenehexamine (PEHA), hexaethyleneheptamine (HEHA), and higher molecular weight materials with a number-average molecular weight of 250 to 300 grams per mole, available from Huntsman. Under a stream of Argon, the temperature in the kettle was raised to 100° C. and the resin was allowed to melt. When the resin was completely melted, the temperature was gradually raised to 180° C. with stirring, and the reaction was allowed to proceed for 3 hours. 3.6 milliliters of water was collected into the Dean-Stark trap. The reaction was stopped, cooled down to 140° C. and discharged to an aluminum tray to give 249 g of the amide as a beige solid.

A dispersant prepared as described in Example 4 of U.S. Pat. No. 6,309,453, is prepared as follows. About 80.0 grams (0.052 moles) of ARCOL LHT 112 (glycerol propoxylate available from ARCO Chemical Co.) and about 46.6 grams (0.156 moles) octadecyl isocyanate (Mondur O-Octadecyl Isocyanate available from Bayer Corporation) were placed in a 200 milliliter beaker with a magnet and heated to 115° C. with a silicone oil bath. Five drops of catalyst (Fascat® 4202, dibutyltindilaurate available from Elf Atochem North American, Inc.) were added and the mixture allowed to react for 2 hours at 115° C. An FT-IR of the reaction product showed the absence (disappearance) of a peak at ˜2285 cm⁻¹ (NCO) and the appearance (or increase in magnitude) of peaks at ˜1740-1680 cm⁻¹ and ˜1540-1530 cm⁻¹ corresponding to urethane frequencies. The final urethane product was then poured into a specimen jar and allowed to cool and harden. This final product was a solid at room temperature characterized by a viscosity of about 15.8 cPs as measured by a Ferranti-Shirley cone-plate viscometer at about 135° C. and a melting point of about 23.8° C. as measured by a Differential Scanning calorimetry using a DuPont 2100 calorimeter at a scan rate of 20° C./minute.

A triamide wax prepared as described in Example II of U.S. Pat. No. 6,860,930, is prepared as follows. To a 1,000 milliliter four-neck round bottom flask fitted with a Trubore stirrer, N₂ inlet, Dean-Stark trap with condenser and N₂ outlet and thermocouple-temperature controller was added 350.62 grams (0.3675 moles) of UNICID® 550 (a mono-acid obtained from Baker-Petrolite Corp., Cincinnati, Ohio, of the formula CH₃(CH₂)ₙCOOH, wherein n has an average value of about 37 and is believed to have a range of from about 34 to about 40) and 0.79 grams of NAUGARD® 524 (antioxidant obtained from Uniroyal Chemical Company, Inc., Middlebury, Conn.). The mixture was heated to 115° C. to melt and stirred at atmospheric pressure under N₂. 51.33 grams (0.1167 moles) of JEFFAMINE® T-403 (mixture of triamines obtained from Huntsman Corporation, Houston, Tex., of the formula

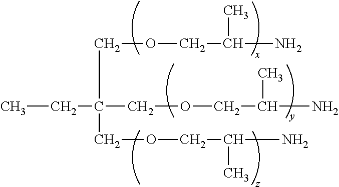

wherein x, y, and z are each integers representing the number of repeat propyleneoxy units, wherein x, y, and z may each be zero, and wherein the sum of x+y+z is from about 5 to about 6) was then added to the reaction mixture, and the reaction temperature was increased to 200° C. gradually over 0.5 hour and held at that temperature for another 3 hours. Some water was carried out by slow blowing N₂ and condensed into the trap when the mixture temperature reached about 180° C. The trap and condenser were then removed, and vacuum (about 25 mm Hg) was applied for about 0.5 hour and then released. The liquid product was cooled down to about 150° C. and poured onto aluminum to solidify. The resulting product was believed to be of the formula

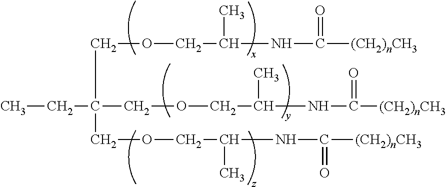

wherein n, x, y, and z are as defined hereinabove in this Example.

### Example 1

Compound 1, a modified indigo carmine, was prepared as follows. To a 1,000 milliliter beaker fitted with magnetic stirring and heating were introduced 500 milliliters deionized water and 15 grams (0.032 mol) of Indigo Carmine. The temperature was raised to 80° C. and 47.4 grams (0.076 mol) of N,N-dimethyldioctadecylammonium bromide were added to the solution. The mixture was heated at 80° C. for 2 hours. The purple solid was isolated via filtration using a glass frit while the mixture was still hot. After the product was allowed to air dry, 43 grams (90%) of a purple powder were obtained.

### Example 2

Compound 2, a modified indigo carmine was prepared as follows. To a 500 milliliter beaker fitted with magnetic stirring and heating were introduced 200 milliliters deionized water and 5 grams (0.011 mol) of Indigo Carmine. The temperature was raised to 80° C. and 7.81 grams (0.021 mol) of cetyltrimethylammonium bromide were added to the solution. The mixture was heated at 80° C. for 2 hours. The purple solid was isolated via filtration using a glass frit while the mixture was still hot. After the product was allowed to air dry, 9.8 grams (92%) of a dark purple powder were obtained.

### Example 3

Compound 3, a modified Indigo Carmine with Arquad® 316, was prepared as follows. To a 1000 milliliter beaker fitted with magnetic stirring and heating were introduced 500 milliliters deionized water and 15 grams (0.032 mol) of Indigo Carmine. The temperature was raised to 80° C. and 23.7 grams (0.038 mol) of Arquad® 316 were added to the solution. The mixture was heated at 80° C. for 2 hours. The purple solid was isolated via filtration using a glass frit while the mixture was still hot. The compound was washed on the frit twice with 200 milliliter distilled water. After the product was allowed to air dry, 27.5 grams (75%) of a violet powder were obtained.

### Example 4

Compound 4, a modified Alizarin Red S, was prepared as follows. Into a 500 milliliter beaker fitted with magnetic stirring, heating mantel were introduced 250 milliliters distilled water, 8 grams (0.023 mol) Alizarin Red S. Once the Alizarin Red S was dissolved, to the solution were added 16.9 grams (0.023 mol) Arquad® 316. The temperature was raised to 80° C. and the mixture was allowed to stir for an hour. Most of the liquid was decanted and the remaining gel was taken in 300 milliliter hexanes. The resultant water was separated using a separatory funnel. The resultant yellow hexanes solution was dried over magnesium sulfate, and the liquid was separated by filtration using a Buchner funnel. After the removal of the solvent ion vacuo, 11.52 grams (49%) of a dark green pasty solid were obtained.

### Example 5

Pigmented Solid Ink Containing Modified Indigo Carmine Compound 1 and a dispersant as described in Example 1 of U.S. Pat. No. 7,973,186. The following materials were weighed in a 600 milliliter beaker such that the accompanying weight percentages also include the pigment and dispersant that were added in subsequent mixing step: 80.12 grams (50.08%) of polymethylene wax, 23.1 grams (14.44%) triamide wax, 24 grams (15%) of a KEMAMIDE® S180, 23.1 grams (14.44%) KE-100 resin, 4 grams (2.5%) urethane resin, 0.496 grams (0.31%) NAUGARD® 445, and 2.304 grams (1.44%) of dispersant (prepared as described in Example 1 of U.S. Pat. No. 7,973,186, which is hereby incorporated by reference herein in its entirety). The materials were melted in an oven at 120° C., mixed well, then transferred to a Szegvari 01 attritor, available from Union Process, that was also heated to 120° C., and charged with 1,800 grams 440 C type ⅛ inch diameter stainless steel balls available from Hoover Precision Products. A heated impeller was attached to the assembly whereupon the impeller speed was adjusted such that the stainless steel balls at the top of the vessel began to tumble gently over each other. To this stirring solution were added 2.88 grams (1.8%) of Modified Indigo Carmine Compound 1 of Example 1. The pigmented ink was allowed to attrite at 300 revolutions per minute (RPM) for 20 hours upon which the final attrited mixture was isolated from the stainless steel balls and filtered with a 5 micron stainless steel mesh using a KST-47 filtration apparatus, commercially available from Advantec MFS. Inc.

### Example 6

Pigmented Solid Ink Containing Modified Indigo Carmine Compound 1 and dispersant as described in Example 1 of U.S. Pat. No. 7,973,186. The Ink of Example 2 was prepared as in Example 1 except that the milling time was increased from 20 hours to 42 hours.

### Example 7

Pigmented Solid Ink containing Modified Indigo Carmine Compound 1 and Solsperse® 17000. The following materials were weighed in a 600 milliliter beaker such that the accompanying weight percentages also include the pigment and dispersant that were added in subsequent mixing step: 80.12 grams (50.08%) of polymethylene wax, 23.1 grams (14.44%) triamide wax, 24 grams (15%) of KEMAMIDE® S180, 23.1 grams (14.44%) KE-100 resin, 4 grams (2.5%) urethane resin, 0.496 grams (0.31%) NAUGARD 445 and 2.304 grams (1.44%) of SOLSPERSE® 17000. The materials were melted in an oven at 120° C., mixed well, then transferred to a Szegvari 01 attritor, available from Union Process, that was also heated to 120° C., and charged with 1800 grams 440 C type ⅛ inch diameter stainless steel balls available from Hoover Precision Products. A heated impeller was attached to the assembly whereupon the impeller speed was adjusted such that the stainless steel balls at the top of the vessel began to tumble gently over each other. To this stirring solution were added 2.88 grams (1.8%) of Modified Indigo Carmine Compound 1 of Example 1. The pigmented ink was allowed to attrite at 300 RPM for 20 hours upon which the final attrited mixture was isolated from the stainless steel balls and filtered with a 5 micron stainless steel mesh using a KST-47 filtration apparatus, commercially available from Advantec MFS. Inc.

### Example 8

Pigmented Solid Ink containing Modified Alizarin Red S Compound 4. A molten and thoroughly mixed blend consisting of parts (84.7 grams) (52.94%) of a polymethylene wax, 23.7 grams (14.82%) triamide wax, 21.5 grams (13.42%) KE-100 resin, 22.8 grams (14.25%) KEMAMIDE® S180, 1.44 grams (0.9%) urethane resin, and 0.256 grams (0.17%) NAUGARD® 445 were placed into a 600 milliliter beaker on top of a hot plate and allowed to stir for 1 hour at 120° C. To this were slowly added, 5.616 grams (3.51%) Modified Alizarin Red S Dye Compound 4 of Example 4. The resultant ink was stirred for 2.5 hours at 120° C. and then was filtered through a 5 μm stainless steel mesh.

### Example 9

Pigmented Solid Ink containing Modified Indigo Carmine Compound 2 and dispersant as described in Example 1 of U.S. Pat. No. 7,973,186. The following materials were weighed in a 600 milliliter beaker such that the accompanying weight percentages also include the pigment and dispersant that were added in subsequent mixing step: 80.12 grams (50.08%) of polymethylene wax, 23.1 grams (14.44%) triamide wax, 24 grams (15%) of KEMAMIDE® 5180, 23.1 grams (14.44%) KE-100 resin, 4 grams (2.5%) urethane resin, 0.496 grams (0.31%) NAUGARD® 445, and 2.304 grams (1.44%) of a dispersant (prepared as described in Example 1 of U.S. Pat. No. 7,973,186. The materials were melted in an oven at 120° C., mixed well, then transferred to a Szegvari 01 attritor, available from Union Process, that was also heated to 120° C., and charged with 1800 grams 440 C type ⅛ inch diameter stainless steel balls available from Hoover Precision Products. A heated impeller was attached to the assembly whereupon the impeller speed was adjusted such that the stainless steel balls at the top of the vessel began to tumble gently over each other. To this stiffing solution were added 2.88 grams (1.8%) of Modified Indigo Carmine Compound 2 of Example 2. The pigmented ink was allowed to attrite at 300 RPM for 20 hours upon which the final attrited mixture was isolated from the stainless steel balls and filtered with a 5 micron stainless steel mesh using a KST-47 filtration apparatus, commercially available from Advantec MFS. Inc.

### Example 10

Pigmented Solid Ink containing Modified Indigo Carmine Compound 2 and SOLSPERSE® 17000. The following materials were weighed in a 600 milliliter beaker such that the accompanying weight percentages also include the pigment and dispersant that were added in subsequent mixing step: 80.12 grams (50.08%) of polymethylene wax, 23.1 grams (14.44%) triamide wax, 24 grams (15%) of KEMAMIDE® 5180, 23.1 grams (14.44%) KE-100 resin, 4 grams (2.5%) urethane resin, 0.496 grams (0.31%) NAUGARD® 445, and 2.304 grams (1.44%) of SOLSPERSE® 17000. The materials were melted in an oven at 120° C., mixed well, then transferred to a Szegvari 01 attritor, available from Union Process, that was also heated to 120° C., and charged with 1800 grams 440 C type ⅛ inch diameter stainless steel balls available from Hoover Precision Products. A heated impeller was attached to the assembly whereupon the impeller speed was adjusted such that the stainless steel balls at the top of the vessel began to tumble gently over each other. To this stiffing solution were added 2.88 grams (1.8%) of Modified Indigo Carmine Compound 2 of Example 2. The pigmented ink was allowed to attrite at 300 RPM for 20 hours upon which the final attrited mixture was isolated from the stainless steel balls and filtered with a 5 micron stainless steel mesh using a KST-47 filtration apparatus, commercially available from Advantec MFS. Inc.

### Comparative Example 11

Pigmented Solid Ink containing Alizarin Red S from Sigma-Aldrich®. The following materials were weighed in a 600 milliliter beaker such that the accompanying weight percentages also include the pigment and dispersant that were added in subsequent mixing step: 80.24 grams (50.15%) of polymethylene wax, 23.1 grams (14.4%) triamide wax, 24 grams (15%) of KEMAMIDE® 5180, 23.1 grams (14.4%) KE-100 resin, 4 grams (2.5%) urethane resin, 0.496 grams (0.31%) NAUGARD® 445, and 2.304 grams (1.44%) SOLSPERSE® 17000. The materials were melted in an oven at 120° C., mixed well, then transferred to a Szegvari 01 attritor, available from Union Process, that was also heated to 120° C., and charged with 1800 grams 440 C type ⅛ inch diameter stainless steel balls available from Hoover Precision Products. A heated impeller was attached to the assembly whereupon the impeller speed was adjusted such that the stainless steel balls at the top of the vessel began to tumble gently over each other. To this stiffing solution were added 2.88 grams (1.8%) of Alizarin Red S as obtained from Sigma-Aldrich®. The pigmented ink was allowed to attrite at 300 RPM for 20 hours upon which the final attrited mixture was isolated from the stainless steel balls and filtered with a 5 micron stainless steel mesh using a KST-47 filtration apparatus, commercially available from Advantec MFS. Inc.

### Comparative Example 12

Pigmented Solid Ink containing Indigo Carmine from Sigma-Aldrich® and SOLSPERSE® 17000. The following materials were weighed in a 600 milliliter beaker such that the accompanying weight percentages also include the pigment and dispersant that were added in subsequent mixing step: 80.12 grams (50.08%) of polymethylene wax, 23.1 grams (14.44%) triamide, 24 grams (15%) of KEMAMIDE® S 180, 23.1 grams (14.44%) KE-100, 4 grams (2.5%) urethane resin, 0.496 grams (0.31%) NAUGARD® 445 and 2.304 grams (1.44%) SOLSPERSE® 17000. The materials were melted in an oven at 120° C., mixed well, then transferred to a Szegvari 01 attritor, available from Union Process, that was also heated to 120° C., and charged with 1800 grams 440 C type ⅛ inch diameter stainless steel balls available from Hoover Precision Products. A heated impeller was attached to the assembly whereupon the impeller speed was adjusted such that the stainless steel balls at the top of the vessel began to tumble gently over each other. To this stiffing solution were added 2.88 grams (1.8%) of Indigo Carmine as obtained from Sigma-Aldrich®. The pigmented ink was allowed to attrite at 300 RPM for 20 hours upon which the final attrited mixture was isolated from the stainless steel balls to be filtered. The ink did not go through the filter at all (not even one gram) at 1.5 pounds per square inch (psi). Although the applied pressure was increased to more than 10 psi, the ink could not be filtered. It was apparent that a gel layer had formed on top of the stainless steel mesh during filtration impeding the flow of ink through filter.

It will be appreciated that various of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims. Unless specifically recited in a claim, steps or components of claims should not be implied or imported from the specification or any other claims as to any particular order, number, position, size, shape, angle, color, or material.

1. A modified naturally-derived colorant comprising:
a naturally-derived colorant that is modified with an aliphatic quaternary salt, an olephenic quaternary salt, an aromatic quaternary salt, or a mixture or combination thereof.

2. The modified naturally-derived colorant of claim 1, wherein the modified naturally-derived colorant is compatible with phase change ink vehicles.

3. The modified naturally-derived colorant of claim 1, wherein the naturally-derived colorant is derived from a member of the group consisting of tetrapyrroles, tetra-terpenoids, quinines, O-heterocyclic compounds, N-heterocyclic compounds, metallo-proteins, lipofuscins, and fungal pigments.

4. The modified naturally-derived colorant of claim 1, wherein the naturally-derived colorant is derived from a member of the group consisting of Safflower, Caesalpina, Maddar, Kermes, Drago tree, Daemonorops, Cochineal, Lac, Bougainvillea, Golden rod, Teak, Marigold, Weld, Saffron, Parijata, Indigo, Woad, Suntberry, Pivet, molluscs, Murasaki, Water lily, Tulsi, Canna, Lily, Nettles, Balsam, Dahlia, Annatto, Balsam, Blackberries, Lac, Alder, Rofblamala, Custard apple, Harda, and mixtures and combinations thereof.

5. The modified naturally-derived colorant of claim 1, wherein the naturally-derived colorant is derived from a compound of the formula:

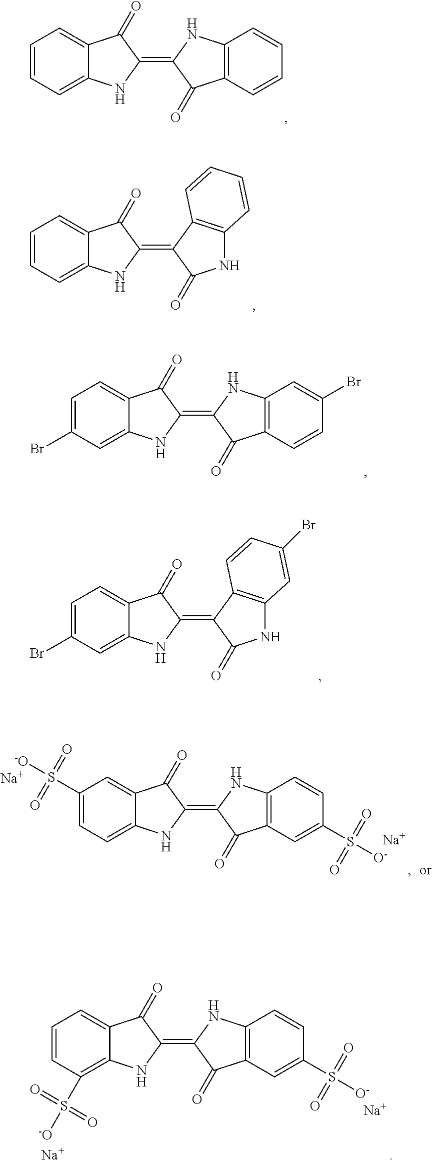

6. The modified naturally-derived colorant of claim 1, wherein the naturally-derived colorant is derived from a compound of the formula:

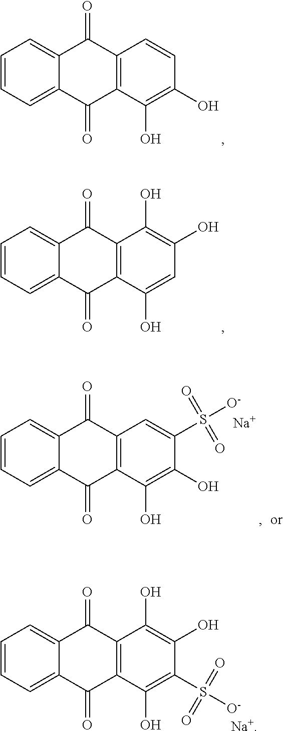

7. The modified naturally-derived colorant of claim 1, wherein the naturally-derived colorant is derived from an anthraquinoid of the formula 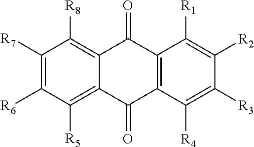
wherein R₁, R₂, R₃, R₄, R₅, R₆, and R₈ are each independently selected from the group consisting of hydrogen, CH₃, OH, and COOH; and
wherein R₇ is selected from the group consisting of hydrogen, a compound of the formula 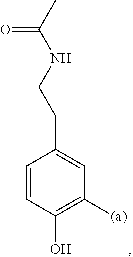
a compound of the formula 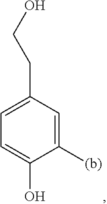
and
a compound of the formula

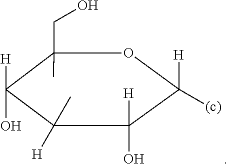

8. The modified naturally-derived colorant of claim 1, wherein the naturally-derived colorant is derived from a compound of the formula

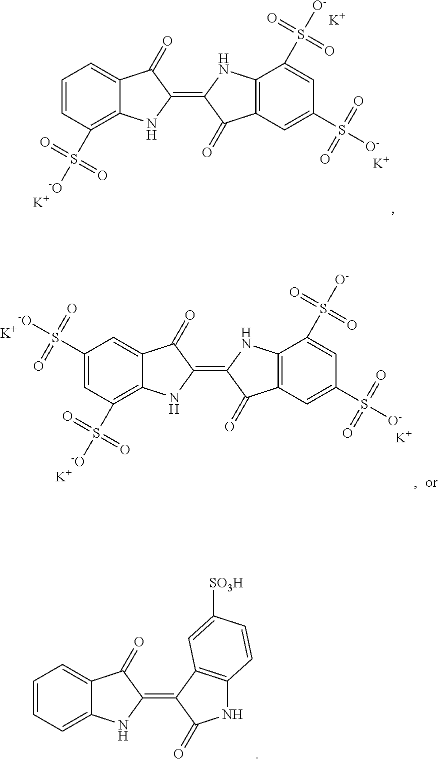

9. The modified naturally-derived colorant of claim 1 wherein the naturally-derived colorant is modified with an aliphatic quaternary ammonium salt, an aromatic quaternary ammonium salt, or a mixture or combination thereof.

10. The modified naturally-derived colorant of claim 1, wherein the naturally-derived colorant is modified with an aliphatic quaternary ammonium salt comprising an alkyl chain having at least eight carbon atoms.

11. The modified naturally-derived colorant of claim 1, wherein the naturally-derived colorant is modified with an aliphatic quaternary ammonium salt or aromatic quaternary ammonium salt selected from the group consisting of tetrabutylammonium, tetraoctyl ammonium, tetradodecylammonium, tetraoctadecylammonium, N,N-dimethyl dioctadecylammonium, N,N-dimethyl dioctyl ammonium, N,N-dimethyl dodecyl ammonium, N,N,N-trimethyl-1-docosanaminium, behenyl trimethylammonium, N-octadecyltrimethylammonium, and mixtures thereof.

12. The modified naturally-derived colorant of claim 1, wherein the naturally-derived colorant is modified with a compound of the formula
R—N⁺(CH₃)₃X⁻
wherein R is a long chain alkyl group having at least 8 carbon atoms and X is a halogen.

13. The modified naturally-derived colorant of claim 1, wherein the naturally-derived colorant is modified with a compound selected from the group consisting of cocoalkyltrimethylammonium, didecyldimethylammonium, coco(fractionated)dimethylbenzylammonium, hexadecyltrimethylammonium, stearyltrimethylammonium, behenyltrimethylammonium, and salts thereof.

14. The modified naturally-derived colorant of claim 1, wherein the naturally-derived colorant is modified with an N-alkyl or N-aryl counterion of the formula 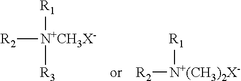
wherein R₁, R₂ and R₃ can be identical or different from one another and wherein each of R₁, R₂ and R₃ is independently selected from the group consisting of alkyl, alkoxy, aryl, and alkylaryl and wherein X is any halogen atom.

15. The modified naturally-derived colorant of claim 1, wherein the naturally-derived colorant is modified with a compound of the formula 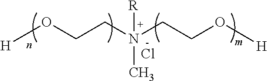
wherein R is H, CH₃, linear alkyl, branched alkyl, or alkoxyl; and wherein m and n are integers and wherein m+n ranges from 2 to 25.

16. The modified naturally-derived colorant of claim 1, wherein the naturally-derived colorant is modified with a compound of the formula 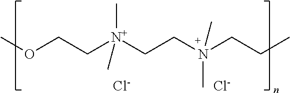
wherein n is from about 1 to about 30.

17. The modified naturally-derived colorant of claim 1, wherein the modified naturally-derived colorant is a compound of the formula

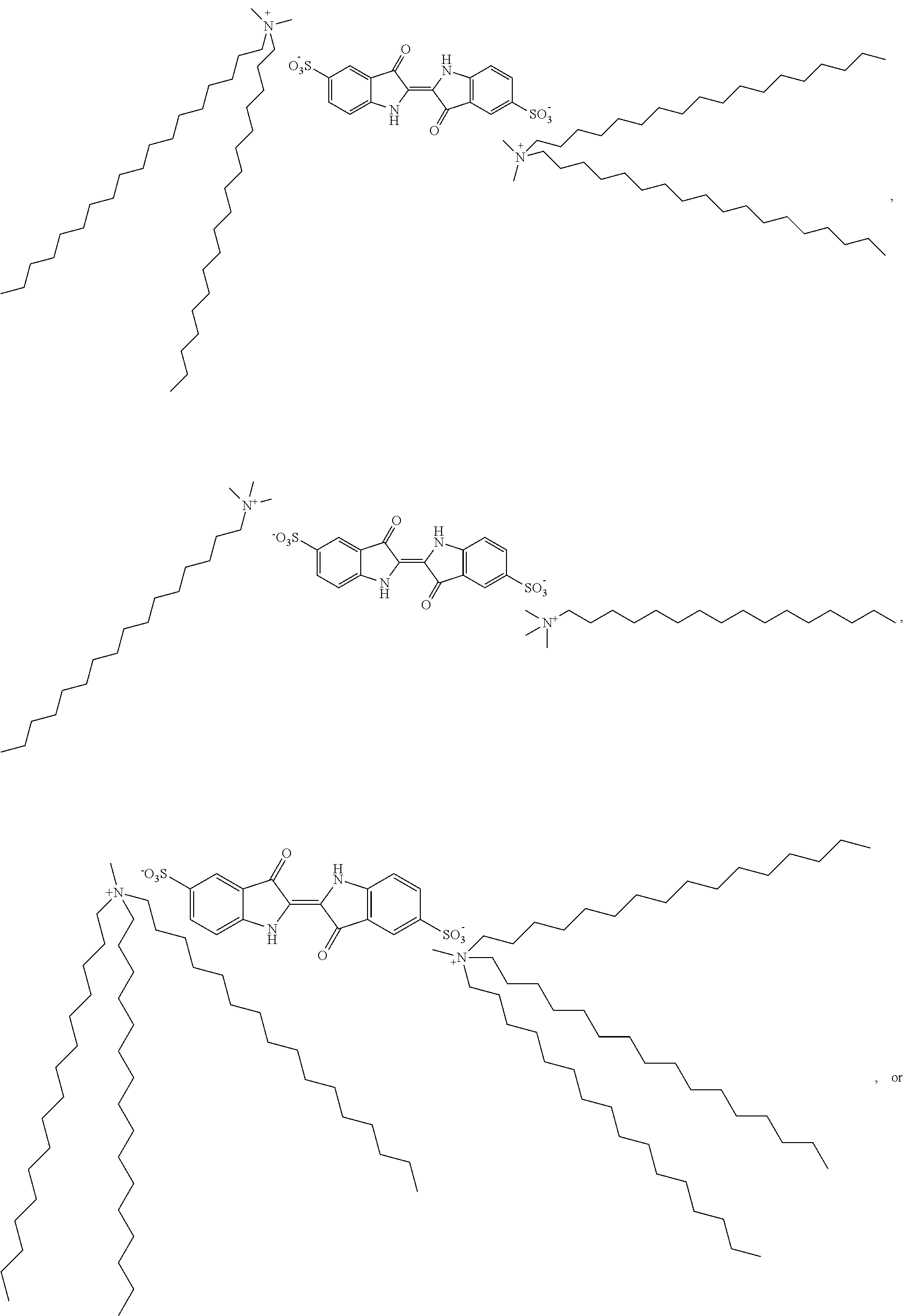

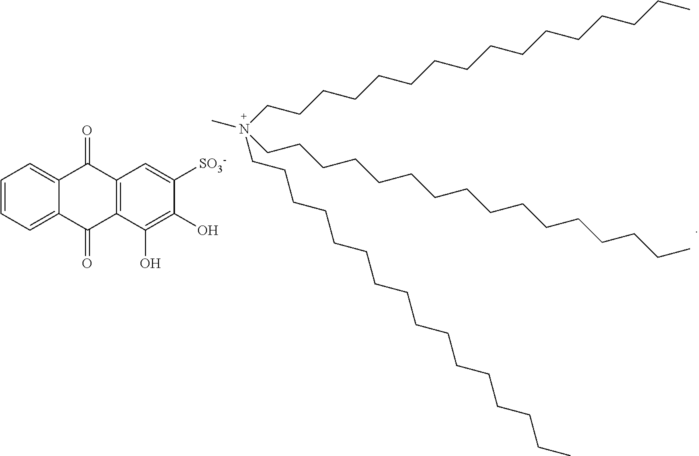

18. The modified naturally-derived colorant of claim 1, wherein the modified naturally-derived colorant is modified with an aliphatic quaternary ammonium salt, aromatic quaternary ammonium salt, or mixture or combination thereof, and wherein the aliphatic quaternary ammonium salt, aromatic quaternary ammonium salt, or mixture or combination thereof is biobased.

19. The modified naturally-derived colorant of claim 1, wherein the modified naturally-derived colorant is modified with an aliphatic quaternary ammonium salt, an aromatic quaternary ammonium salt, or mixture or combination thereof, wherein the aliphatic quaternary ammonium salt, the aromatic quaternary ammonium salt, or mixture or combination thereof contains an alkyl chain having more than 8 carbon atoms, and wherein the alkyl chain is biobased.

20. The modified naturally-derived colorant of claim 1, wherein the modified naturally-derived colorant of is self-dispersible in a phase change ink vehicle.
